(19) United States
(12) Patent Application Publication
(10) Pub. No.: US 2025/0123230 A1
(43) Pub. Date: Apr. 17, 2025

(54) TEST DEVICE AND METHOD FOR MEASUREMENT OF AERATION IN A FLOW STREAM VIA ELECTRICAL PROPERTIES

(71) Applicant: Southwest Research Institute, San Antonio, TX (US)

(72) Inventors: Cole T. FRAZIER, San Antonio, TX (US); Marshall Cole HUDSON, San Antonio, TX (US); Nolan A. ERICKSON, San Antonio, TX (US)

(21) Appl. No.: 18/913,211

(22) Filed: Oct. 11, 2024

Related U.S. Application Data
(60) Provisional application No. 63/590,573, filed on Oct. 16, 2023.

(51) Int. Cl. G01N 27/20; G01N 27/07; G01N 27/08; G01N 27/22; G01N 27/24

(52) U.S. Cl. CPC G01N 27/20; G01N 27/07; G01N 27/08; G01N 27/226; G01N 27/24

(57) ABSTRACT

A device and method for measurement of aeriation in a flow stream via electrical properties. In particular, the device and method allow for the determination of aeration in fluids used in electrical drive type applications.

### FIELD

The present invention is directed to a device and method for measurement of aeriation in a flow stream via electrical properties. In particular, the device and method allow for the determination of aeration in fluids used in electrical drive type applications.

### BACKGROUND

Fluids utilized in electrical drive applications can have varying levels of aeration. Such fluids may include automatic transmission fluid (ATF) and gear lubricants. Aeration can cause problems and adversely affect the intended lubricating purpose of the fluids in the vehicle, as well as adversely impact other components such as fluid pumps. Aeration will also typically increase under relatively high fluid flows. Accordingly, a need exists to provide a reliable testing device and method to evaluate the level of aeration in fluids. In particular, the fluids utilized in electric vehicles, via measurement of electrical properties, at selected flow rates.

### SUMMARY

A device for measuring the aeration level of a fluid comprising a fluid entrance and fluid exit, a first dielectric separator wherein fluid can impinge upon said first dielectric separator and proceed into an annular space and a second dielectric separator that merges fluid received from said annular space when exiting said device. The device also includes a cylindrical shaped outer electrode having an inner diameter and a cylindrical shaped inner electrode having an outer diameter wherein the annular space for fluid flow is provided between the outer electrode inner diameter and the inner electrode outer diameter. The outer electrode and the inner electrode provide a cell constant value in the range of 0.1 to 0.001 and the electrodes are configured to measure the conductivity or capacitance of the fluid to determine the aeration level of such fluid.

A method for measuring the aeration level of a fluid which comprises supplying a device having a fluid entrance and fluid exit, a first dielectric separator wherein fluid can impinge upon said first dielectric separator and proceed into an annular space and a second dielectric separator that merges fluid received from said annular space when exiting said device. The device also includes a cylindrical shaped outer electrode having an inner diameter and a cylindrical shaped inner electrode having an outer diameter wherein the annular space for fluid flow is provided between the outer electrode inner diameter and the inner electrode outer diameter. The outer electrode and the inner electrode provide a cell constant value in the range of 0.1 to 0.001 and the electrodes are configured to measure the conductivity or capacitance of the fluid to determine the aeration level of such fluid. One then introduces fluid to the space between the electrodes and measures the conductivity or capacitance of the fluid in the annular space between the electrodes and determines the level of aeration of the fluid.

### BRIEF DESCRIPTION OF THE DRAWINGS

FIG. 1 provides a cross-sectional view of the preferred device for measurement of electrical properties and corresponding aeration levels of a fluid.

### DETAILED DESCRIPTION

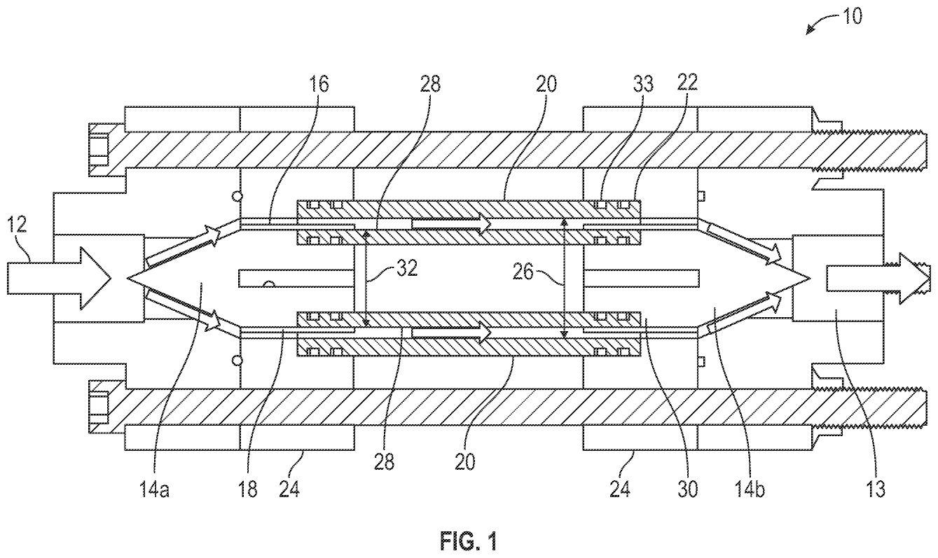

FIG. 1 provides a cross-sectional view of a preferred device 10 herein for measurement of electrical properties and corresponding aeration levels of a given fluid which can be achieved at selected flow rates. The device 10 provides a fluid entrance location 12 for a fluid to flow into the device and a fluid exit location 13 for fluid to exit the device. As can be seen in cross-section, the fluid entering the device impinges upon one or more dielectric separators, which may preferably include a first dielectric separator 14a which preferably has the illustrated diverting-head shape. The incoming fluid after impingement upon the dielectric separator 14a proceeds into the annular space 16 and 18 that is provided between the inner electrode 28 and outer electrode 20.

A second dielectric separator 14b allows for the received fluid to merge at the exit location. The dielectric separator may therefore be described as providing two end portions wherein each end portion preferably has a conical shape. The first and second dielectric separators 14a and/or 14b are preferably prepared from a thermoplastic material, and a particular preferred material comprises poly(etheretherketone) or PEEK.

A cylindrical shaped first outer electrode is shown in cross-section at 20 preferably positioned within a recess portion 22 of the supporting columns 24. The first outer electrode has an inner diameter identified by arrow 26. A cylindrical shaped second inner electrode is shown in cross-section at 28 where a portion of the second electrode is preferably contained in a recess 30 in the dielectric separators 14a and 14b. The first outer electrode and second inner electrode may therefore be present as concentric cylinders which as noted above, define annular space 16 and 18 for fluid flow.

The second inner electrode 28 has an outer diameter identified by arrow 32. O-ring seals can be seen at 33. As may now be appreciated, fluid entering the device impinges upon the dielectric separator 14a and then travels in the annular space present between the outer diameter 32 of the inner electrode 28 and the inner diameter 26 of the of the outer electrode 20. A preferred value for the distance between the electrodes is nominally 0.10 inches. In addition, preferably, the cross-sectional area of the space between the electrodes, that defines the annular space or flow path for fluid, is such that it does not vary by more than +/−5.0% or less. Therefore, preferably, the cross-sectional area of the annular space between the electrodes that defines the flow path does not vary by more than +/−5.0%, or +/−4.0%, or +/−3.0%, or +/−2.0%, or +/−1.0%.

Preferably, the inner electrode 28 and outer electrode 20 are made of stainless steel and as noted, preferably have a concentric cylindrical shape and have a preferred length of 2.0 inches to 10.0 inches, including all values and increments therein. The electrode surface roughness of the outer electrode 20 and inner electrode 28, that are exposed herein to fluid flow, are also preferably characterized as having a Ra value of 3.0 μin to 5.0 μin, including all values and increments therein. More specifically, the dimensional characteristics of the outer electrode 20 and inner electrode 28 are better understood herein with reference to the cell constant values they provide, which is reference to the distance between the outer and inner electrodes divided by the electrode surface area. Accordingly, the cell constants values herein for the subject electrodes is such that it preferably falls in the range of 0.1 to 0.001, including all values and increments therein.

The fluids that may be evaluated in the device 10 for their respective aeration characteristics preferably include fluids that have a viscosity in the range of 4 cSt to 100 cSt. The fluids are also contemplated to preferably include those fluids that are utilized in the transportation sector, e.g., driveline lubricants, engine lubricants, hydraulic lubricants, gear lubricants and automatic transmission fluids. The fluids are also passed through the device with a flow velocity that preferably falls in the range of 2.0 liters/min. to 30.0 liters/min., including all values and increments therein. The flow velocity is also preferably maintained as relatively constant, meaning that the selected flow velocity of the fluid through device 10 does not vary by more than +/−5.0%. Therefore, the flow velocity of the fluid passing through device 10, and undergoing evaluation for its aeration properties, does not vary by more than +/−5.0%, or +/−4.0%, or +/−3.0%, or +/−2.0%, or +/−1.0%.

It may therefore now be appreciated that with respect to any selected fluid herein passing though the device 10, the conductivity of the unaerated fluid can be determined, and the conductivity of air can be determined. Accordingly, the conductivity of a fluid with a given level of aeration will fall somewhere between these two determined values, and the aeration level may be determined from such conductivity measurements. In the broad context of the present invention, the preferred conductivity values that are contemplated for measurement will fall in the range of 1×10⁻¹⁵ S/m to 1×10⁻⁴ S/m, where S/m is reference to siemens per meter. In addition, the capacitance of unaerated fluid and capacitance of air can be determined, and the capacitance of a fluid with a give level of aeration is again contemplated to fall somewhere between the two capacitance values, and the aeration levels may again be determined from such capacitance values. In the broad context of the present invention, the contemplated and preferred capacitance values for measurement will fall in the range of 1 pF to 100 pF, where pF is reference to picofarads.

As may now be appreciated, the device 10 of the present invention provides for a method to determine the aeration level of a fluid via evaluation of electrical properties (conductivity or capacitance). The device and method herein rely upon appropriately sized and controlled inner and outer electrodes (cell constants in the range of 0.1 to 0.001) such that the cross-sectional area of the annular flow path does not vary by more than +/−5.0%, and the fluid flow velocity remains relatively constant within the device and does not vary by more than +/−5.0%, +/−4.0%, +/−3.0%, +/−2.0% or +/−1.0%. This then is contemplated to provide relatively more precise evaluation of electrical properties of the fluids and relatively more accurate evaluation of fluid aeration levels.

What is claimed is:

1. A device for measuring the aeration level of a fluid comprising:
a fluid entrance and fluid exit;
a first dielectric separator wherein fluid can impinge upon said first dielectric separator and proceed into an annular space and a second dielectric separator that merges fluid received from said annular space when exiting said device;
a cylindrical shaped outer electrode having an inner diameter and a cylindrical shaped inner electrode having an outer diameter wherein said annular space for fluid flow is provided between said outer electrode inner diameter and said inner electrode outer diameter;
said outer electrode and said inner electrode provide a cell constant value in the range of 0.1 to 0.001; and
wherein said electrodes are configured to measure the conductivity or capacitance of said fluid to determine the aeriation level of said fluid.

2. The device of claim 1, wherein said annular space for fluid flow defines a cross-sectional area that does not vary by more than +/−5.0%.

3. The device of claim 1, wherein said electrodes measure a conductivity in the range of 1×10⁻¹⁵ S/m to 1×10⁻⁴ S/m.

4. The device of claim 1, wherein said electrodes measure a capacitance in the range of 1 pF to 100 pF.

5. The device of claim 1, wherein said first and second dielectric separators are composed of poly(etheretherketone).

6. The device of claim 1, wherein said annular space has a cross-sectional area that does not vary by more than +/−5.0%.

7. A method for measuring the aeration level of a fluid comprising:
supplying a device having
a fluid entrance and fluid exit, a first dielectric separator wherein fluid can impinge upon said first dielectric separator and proceed into an annular space and a second dielectric separator that merges fluid received from said annular space when exiting said device;
a cylindrical shaped outer electrode having an inner diameter and a cylindrical shaped inner electrode having an outer diameter wherein said annular space for fluid flow is provided between said outer electrode inner diameter and said inner electrode outer diameter;
said outer electrode and said inner electrode provide a cell constant value in the range of 0.1 to 0.001;
introducing fluid to said annular space between said electrodes;
measuring the conductivity or capacitance of said fluid in said annular space between said electrodes and determining the level of aeration of said fluid.

8. The method of claim 7, wherein said annular space for fluid flow defines a cross-sectional area that does not vary by more than +\−5.0%.

9. The method of claim 7, wherein said electrodes measure a conductivity in the range of 1×10⁻¹⁵ S/m to 1×10⁻⁴ S/m.

10. The method of claim 7, wherein said electrodes measure a capacitance in the range of 1 pF to 100 pF.

11. The method of claim 7, wherein said fluid introduced into said device has a viscosity in the range of 4 cSt to 100 cSt.

12. The method of claim 7, wherein said fluid flow has a velocity that does not vary by more than +/−5.0%.

13. The method of claim 7, wherein said first and said second dielectric separators are composed of poly(etheretherketone).
